(19) United States
(12) Patent Application Publication
(10) Pub. No.: US 2026/0020936 A1
(43) Pub. Date: Jan. 22, 2026

(54) OPTIMIZED DENTAL IMPLANT PLACEMENT USING LIBRARY-BASED COMPUTER ALGORITHMS

(71) Applicant: EXOCAD GMBH, Darmstadt (DE)

(72) Inventor: Gustavo Ismael, Sao Paulo (BR)

(21) Appl. No.: 18/777,166

(22) Filed: Jul. 18, 2024

(51) Int. Cl. A61C 7/00; G06T 7/00; G06T 19/00

(52) U.S. Cl. CPC A61C 7/002; G06T 7/0012; G06T 19/003; G06T 2207/30036

(57) ABSTRACT

Disclosed is a computer-implemented method for determining dental implant positions for insertion into a patient’s jaw. The method comprises receiving a three-dimensional digital jaw model and receiving a number of dental implants to be inserted. A preset indicator, which selects an implant library preset based on the number of implants, is also received. These implant library presets, assigned to specific numbers of implants, comprise area indicators for optimal jaw areas for implant insertion. Using the selected implant library preset and the digital jaw model, a set of dental implant parameters defining the positions of the implants relative to the jaw model is determined.

### FIELD OF THE INVENTION

The invention relates to the field of determining dental implant positions of dental implants to be inserted into a patient’s jaw.

### BACKGROUND

Dental implants have become a widely accepted solution for replacing missing teeth, offering numerous benefits over traditional dentures and bridges. These implants, which act as artificial tooth roots, are typically made from biocompatible materials such as titanium or zirconia. They are surgically placed into the jawbone, where they integrate with the bone tissue in a process known as osseointegration. This provides a stable foundation for various types of dental prosthetics, including crowns, bridges, and dentures, ultimately restoring both the functionality and aesthetics of a patient's smile.

The successful placement of dental implants requires careful planning and precise execution. Determining the optimal positions for implants involves assessing multiple factors, including the quality and quantity of the jawbone, the spatial relationship with existing teeth, and the proximity to critical anatomical structures such as nerves and sinus cavities. Accurate placement is crucial to ensure the longevity and stability of the implants, as well as to avoid potential complications that could arise from improper positioning.

Traditionally, determining dental implant positions involves a detailed process relying on the expertise of dental professionals. It starts with a thorough clinical examination and traditional two-dimensional radiographs, such as panoramic X-rays, to assess the jawbone and identify anatomical structures to avoid. Dental impressions are taken to create physical models of the patient’s mouth, which serve as references for planning implant placement.

Diagnostic wax-ups and surgical stents are then used to visualize the final restoration and guide the implant placement during surgery. In advanced cases, CT scans provide detailed images of the jawbone, aiding in more precise planning, though interpretation still relies heavily on clinician judgment.

Manual measurements and meticulous planning are crucial to ensure implants are correctly spaced, aligned with adjacent teeth, and properly integrated with the patient’s bite. Despite the use of advanced imaging tools, traditional methods remain labor-intensive, time-consuming, and highly dependent on the skill of the dentist, with a higher potential for human error impacting the overall success of the dental implants.

### SUMMARY OF THE INVENTION

It is an objective to provide for an improved computer-implemented method, an improved computer program and an improved computer device for determining one or more dental implant positions for one or more dental implants to be inserted into a patient’s jaw as well as an improved manufacturing system comprising a manufacturing device configured to manufacture a physical drilling template. The objectives underlying the invention are solved by the features of the independent claims.

In one aspect, a computer-implemented method for determining one or more dental implant positions for one or more dental implants to be inserted into a patient’s jaw is disclosed. The method comprises receiving a three-dimensional digital jaw model of the patient’s jaw. A number of dental implants to be inserted into the patient’s jaw is received. A preset indicator indicating an implant library preset selected from a plurality of implant library presets depending on the number of implants is received. The implant library presets are assigned to respective numbers of implants. The plurality of implant library presets comprise area indicators of jaw areas for insertion of the respective numbers of dental implants. A set of dental implant parameters defining one or more dental implant positions relative to the three-dimensional digital jaw model are determined using the selected implant library preset and the three-dimensional digital jaw model.

For example, dental implants comprise artificial tooth roots. The dental implants may be constructed from a group of biocompatible materials, the group of biocompatible materials may comprise titanium and zirconia. For example, the dental implants are designed to be surgically placed into the patient’s jawbone. These implants could serve as a foundation for various dental prosthetics, such as crowns, bridges, or dentures.

For example, a jawbone refers to the bony structure that forms the framework of the mouth and supports the teeth. For example, it comprises the maxilla, which is the upper jaw, and the mandible, which is the lower jaw. These bones may be essential for various functions, such as e.g., chewing, speaking, and maintaining the shape of the face.

For example, the jawbone comprises bone tissue and a spongy inner layer that supports the roots of the teeth. For example, the mandible could be the only movable bone of the jaw, allowing for the opening and closing of the mouth, which is crucial for eating and speaking. The maxilla may be fixed and may comprise the upper dental arch, e.g., housing the upper teeth.

The structure of the jawbone might comprise an alveolar ridge, which may be the part of the jawbone that may hold the tooth sockets, and the temporomandibular joint (TMJ), which may connect the mandible to the skull and may allow for jaw movement. Furthermore, the jawbone may comprise canals and foramina that e.g., allow for the passage of nerves and blood vessels, such as the inferior alveolar nerve that may run through the mandible.

The dental implant positions might be the specific locations within the jawbone where these implants could be optimally placed. For example, dental implant positions correspond to positions of the patient’s existing or missing natural teeth. For example, determining these optimal positions involves careful consideration of multiple factors, such as the quality and quantity of the jawbone, the proximity to critical anatomical structures (e.g., nerves and sinus cavities), and the spatial relationship with neighboring teeth. Properly identified implant positions may ensure that the implants integrate well with the bone (osseointegration) and could provide a stable and durable foundation for the attached prosthetics. For example, the dental implant positions are adjusted after the determination by the computer-implemented method. They may be manually adjusted by a person skilled in the art. They may be adjusted by software or computer-implemented methods.

The patient's jaw could refer to either the mandible (lower jaw) or the maxilla (upper jaw). The patient’s jaw may be the structure that supports the patient’s teeth. For example, the patient’s jaw comprises the jawbone. This area could be critically analyzed to identify suitable sites for implant placement. The condition of the patient's jaw might be assessed to e.g., determine if there is sufficient bone density and volume to support the implants. Bone grafting procedures may be required if e.g., bone deficiencies are found.

The three-dimensional digital jaw model may be a detailed and accurate digital representation of the patient's jaw. For example, it is created using advanced imaging technologies. Advanced imaging technologies may comprise computed tomography (CT) scans, cone-beam computed tomography (CBCT) scans and/or three-dimensional intraoral scanners. The three-dimensional digital jaw model may provide a comprehensive view of the jaw’s anatomy, wherein the jaw’s anatomy may comprise bone contours, density, and/or the position of existing and/or missing teeth and/or other relevant anatomical structures. Such a model could be crucial for planning the precise placement of implants, e.g., allowing dental professionals to visualize the jaw in three dimensions and simulate various dental implant placement scenarios.

For example, the number of dental implants defines the total count of implants planned for insertion into the patient’s jaw. For example, a patient who has lost multiple teeth might require several implants to replace each missing tooth or to support a multi-tooth prosthetic like a bridge or denture. This count could be a critical factor in e.g., determining the overall treatment plan, potentially influencing the selection of appropriate implant sizes, types, and placement strategies.

A preset indicator could be a signal or marker that e.g., identifies a specific implant library preset. For example, an implant library preset is a pre-defined set of guidelines, templates, and/or protocols designed to assist in determining the optimal implant positions based on the number of implants needed. For example, if a patient requires X implants, the preset indicator could help select a specific preset tailored for the placement of X implants in the most effective and anatomically suitable positions depending on a variety of factors. The variety of factors may comprise the three-dimensional digital jaw model.

The implant library presets could comprise area indicators of jaw areas, which might be markers or suggested zones within the jaw where the implants could ideally be placed for optimal results. For example, a preset for one or more implants might indicate specific regions of the jaw where the bone is sufficiently dense and voluminous to support the one or more implants, avoiding areas that could be problematic, such as those with insufficient bone or proximity to sensitive anatomical structures. The implant library presets may be adjusted. They may be manually adjusted by a person skilled in the art. They may be adjusted by software and/or computer-implemented methods.

For example, the set of dental implant parameters comprises dental implant positions. For example, the dental implant positions comprise coordinates defining positions of dental implants. For example, the dental implant positions comprising coordinates defining positions of dental implants are relative to the three-dimensional digital jaw model. For example, the dental implant positions further comprise the exact or approximate depth of dental implants, the spacing between dental implants, their alignment with existing teeth, and/or their orientation relative to the three-dimensional jaw model. For example, approximate defines a value comprising a margin of error of 5%, in particular 2%, in particular 0.5%. For example, the orientation is relative to the occlusal plane (the biting surface). By e.g., using the selected implant library preset and the digital jaw model, the dental implant positions could be determined to ensure accurate and effective implant placement. For example, the set of dental implant parameters is adjusted after the determination by the computer-implemented method. They may be manually adjusted by a person skilled in the art. They may be adjusted by software or computer-implemented methods.

The disclosed method for determining dental implant positions using a computer-implemented approach could offer numerous other advantages. Any advantages that are described in this patent application that may result from the dental implanting procedure itself are a result of an improved planning facilitated by the computer-implemented method, the improved computer program and the improved computer device for determining one or more dental implant positions for one or more dental implants to be inserted into a patient’s jaw as well as a result from the improved manufacturing system comprising a manufacturing device configured to manufacture a physical drilling template. Hence, the benefits/advantages stated in this application refer specifically to advantages during the planning stage and are not related to the medical procedure, i.e., the dental implanting procedure. Therefore, any disclosed advantages are not referring to the dental implanting procedure itself.

Enhanced precision may be achieved through the use of a three-dimensional digital jaw model, potentially allowing for highly accurate planning and placement of dental implants. For example, this improved precision during dental implant planning could reduce the risk of misalignment and improve the overall success rate. Customization during dental implant planning is another potential benefit, as the method could enable treatment plans tailored to the specific anatomical structure of each patient’s jaw, potentially leading to better fitting and more comfortable dental prosthetics.

Additionally, the improved method for dental implant planning may subsequently reduce surgical risk by e.g., providing detailed visualizations and precise implant parameters, potentially helping to avoid critical anatomical structures such as nerves and sinus cavities. This improved dental implant planning could significantly lower the risk of complications during surgery. Improved osseointegration might also be a result of optimal implant positioning during dental implant planning, where e.g., the implant fuses effectively with the jawbone, potentially resulting in stronger and more stable implants.

Time efficiency could be another advantage, with the method streamlining the dental implant planning process, allowing for quicker decision-making and reducing the overall time required for treatment planning. Enhanced communication between dental professionals and patients might be facilitated by detailed digital models and preset guidelines, potentially helping patients understand their treatment options and expected outcomes more clearly.

The improved dental implant planning may minimize human error, thereby potentially increasing the reliability of the procedure. For example, this could lead to more predictable outcomes, as pre-defined implant library presets ensure that the implant placement may be consistent with best practices and clinical guidelines. For example, versatility during dental implant planning is another potential benefit, as the method may accommodate a wide range of clinical scenarios and patient needs, including different numbers of implants and varying jaw anatomies, making it a versatile tool in dental implantology.

Improved dental implant planning could improve the aesthetic outcome of dental prosthetics, e.g., ensuring that they look natural and are properly aligned with the existing teeth. For example, this precision reduces the need for revisions, as optimal placement from the outset may be decreasing the likelihood of needing corrective surgeries or adjustments in the future. As a result, patient satisfaction could be higher due to e.g., the combination of reduced surgical risks, improved aesthetics, and predictable outcomes.

For example, improved dental implant planning might also offer cost efficiency by subsequently minimizing the risk of complications and the need for additional corrective procedures, leading to overall cost savings for both dental practices and patients. Comprehensive planning could be another advantage, with the ability to visualize and plan the entire treatment process digitally, e.g., considering all relevant factors to ensure a thorough and well-integrated treatment plan.

Scalability is another potential advantage, as the method could be easily adapted for use in various dental practices, from small clinics to large dental hospitals, ensuring widespread applicability. For example, improved record-keeping practices might result from digital planning and documentation, providing a comprehensive history of the patient’s treatment plan and progress. Patient engagement could be increased by involving patients in the digital planning process, leading to better compliance with pre- and post-operative care instructions.

For example, the implant library presets further comprise one or more definitions of distances between at least two implants.

For example, the definitions of distances between at least two implants comprise the spatial distance between the at least two implants. For example, the definitions of distances may be a range of spatial distances.

These distances could be expressed in millimeters and may specify the minimum and maximum allowable spacing to ensure proper osseointegration and stability. For example, the distance between two implants might be defined as 1 mm to 7 mm, in particular 3 mm to 5 mm.

For example, in case the number of dental implants is higher than two implants, the implant library presets further comprise definitions of distances between each of the implants.

One advantage could be that by ensuring that implants are spaced correctly, the overall success rate of the dental implant procedure may be improved due to the advantages of the improved dental implant planning. For example, proper spacing as a result of the improved dental implant planning could enhance the osseointegration process, where the implants may fuse effectively with the jawbone, potentially resulting in stronger and more stable support for the prosthetics. Additionally, by e.g., maintaining optimal distances as a result of the improved dental implant planning, this could reduce the risk of complications such as bone resorption or implant failure.

For example, the precise placement of implants according to these distance definitions as a result of the improved dental implant planning could also subsequently improve the aesthetic outcome, e.g., by providing a more natural appearance to the dental prosthetics. This improved dental implant planning might thus lead to increased patient satisfaction, as well-placed implants may be less likely to cause discomfort or may not require frequent adjustments. Furthermore, proper spacing as a result of the improved dental implant planning could facilitate easier and more effective post-surgical maintenance, potentially allowing for better access during cleaning and care, which may reduce the risk of peri-implantitis and other complications.

For example, the implant library presets further comprise one or more relative angulations between at least two implants, wherein the determined dental implant parameters further define one or more dental implant angles for the one or more dental implants relative to the three-dimensional digital jaw model.

For example, the relative angulations between at least two implants comprise angular relationships and/or orientations that define how the dental implants should be positioned relative to each other within the patient's jaw. These relative angulations may be expressed in degrees. For example, the relative angulation between two implants is specified as an angle of X degrees or a range of X to Y degrees, indicating that the orientation of the first implant forms an X-degree angle with the orientation of the second implant.

For example, one or more dental implant angles may comprise definitions of specific angles at which the implants are inserted into the jawbone. These angles might be defined relative to the three-dimensional digital jaw model. For example, the dental implant angles may be within a range of 45° to 90° for angled dental implants and/or straight dental implants relative to the occlusal plane. For example, this range may be the angulation range. For example, the dental implant angles are also referenced relative to any other referencing point.

An advantage could be that correct angulation as a result of the improved dental implant planning may enhance the osseointegration process, where the implants may fuse effectively with the jawbone, resulting in e.g., a stronger and more stable foundation for the dental prosthetics. This precise alignment as a result of the improved dental implant planning could reduce the risk of implant failure and could potentially improve the longevity of the prosthetics.

Furthermore, the correct angulation of implants as a result of the improved dental implant planning could improve the aesthetic outcome by e.g., ensuring that the prosthetics align properly with the natural teeth and the overall dental arch as a result of the improved dental implant planning. This might lead to a more natural and visually pleasing result, enhancing patient satisfaction. Properly angled implants as a result of the improved dental implant planning could also distribute bite forces more evenly by e.g., reducing the stress on individual implants and minimizing the risk of complications such as bone resorption or implant fracture.

For example, the computer-implemented method further comprises generating, using the dental implant parameters, an output comprising a virtual implant positioning model. The virtual implant positioning model visually represents the one or more dental implant positions relative to the three-dimensional digital jaw model of the patient’s jaw.

For example, a virtual implant positioning model could show the precise spots within the jawbone where the implants are to be placed. For example, this model comprises various perspectives, such as cross-sectional views and three-dimensional renderings, which illustrate how the implants align with existing teeth, bone structures, and critical anatomical features like nerves and sinus cavities.

The virtual implant positioning model may be a valuable tool for patient education. By e.g., visualizing the implant positions, patients may gain a clear understanding of their treatment plan. For example, a dentist uses the model to show a patient how the implants may be placed, thereby potentially explaining the rationale behind the positioning, and potentially addressing any concerns. This visual aid could enhance communication, potentially making it easier for patients to grasp the complexities of the procedure and the expected outcomes.

Additionally, the virtual implant positioning model may play a crucial role in surgical planning. For example, surgeons could use the model to simulate the procedure, exploring different scenarios and adjusting as necessary before the actual surgery. This pre-surgical visualization could help in identifying potential challenges, potentially optimizing implant placement, and potentially planning the surgical approach in detail.

One advantage could be that the improved dental implant planning comprising the virtual implant positioning model may subsequently enhance precision and accuracy in implant placement, potentially reducing the risk of complications such as misalignment or damage to anatomical structures. This precision could lead to better osseointegration, where the implants may fuse more effectively with the bone, potentially resulting in a more stable and durable foundation for dental prosthetics.

Another advantage could be that the improved dental implant planning comprising the model could improve patient satisfaction by potentially providing a clear and comprehensive understanding of the treatment plan, potentially leading to increased confidence and reduced anxiety about the procedure. Patients who may be well-informed and involved in their treatment decisions may be more likely to adhere to pre- and post-operative care instructions, which could improve overall outcomes.

Furthermore, the virtual implant positioning model may streamline the surgical process, thus e.g., saving time and resources. By e.g., planning the procedure in advance, surgeons may perform the surgery more efficiently, potentially reducing the time patients spend under anesthesia and lowering the risk of surgical errors.

For example, the computer-implemented method further comprises generating, using the dental implant parameters and the three-dimensional digital jaw model, a three-dimensional digital drilling guide model. The three-dimensional digital drilling guide model comprises means for achieving the dental implant parameters for the insertion of the one or more dental implants. The means comprise one or more through holes defining drilling positions of drilling holes to be drilled into the patient’s jaw at the one or more dental implant positions. The drilling positions are to be drilled into patient’s jaw for the insertion of the one or more dental implants.

For example, the computer-implemented method could further involve generating the digital drilling guide model. This model may comprise means for achieving the dental implant parameters, which could refer to the specific guidelines and measurements needed to insert the implants correctly. These means might include features like through holes, which may define the exact drilling positions for creating the necessary drilling holes in the patient’s jaw. For example, the through holes are spaced to achieve a certain dental implant size.

The through holes in the drilling guide model could be strategically placed to e.g., indicate where the dentist should drill into the jawbone. For example, if the dental implant parameters specify that an implant needs to be inserted at a particular angle and depth, the through holes in the guide might be aligned accordingly to facilitate this precise drilling. These holes could act as a template for the surgical drill, e.g., ensuring that the drilling positions match the planned implant locations exactly.

Drilling positions may refer to the specific spots on the jaw where the drill will e.g., penetrate to create the implant sites. These positions could be carefully determined based on the digital jaw model and the implant parameters to avoid critical anatomical structures and to ensure optimal placement for osseointegration.

The drilling holes could be the actual holes drilled into the jawbone, guided by the through holes in the drilling guide model. These holes might be created to the precise depth, angle, size, and position needed to accommodate the dental implants as planned.

One advantage of the three-dimensional digital drilling guide model may be that it may subsequently enhance precision and accuracy in drilling, reducing the risk of errors and complications during surgery. This precision as a result of the improved dental implant planning might lead to better osseointegration, where the implants fuse more effectively with the bone, resulting in a stable and durable foundation for dental prosthetics.

The improved dental implant planning comprising the guide model could further improve patient satisfaction by providing a clear and comprehensive understanding of the treatment plan, leading to increased confidence and cooperation. Well-informed patients might be more likely to follow pre- and post-operative care instructions, e.g., improving overall outcomes.

Additionally, the improved dental implant planning comprising the use of a digital drilling guide model may streamline the surgical process, making it more efficient and potentially reducing the time the patient spends under anesthesia. This efficiency might not only enhance the patient experience but e.g., may subsequently also increase the overall success rate of the implant procedure.

For improved surgical planning, the digital drilling guide model could provide a detailed roadmap for the surgery. Surgeons might use these models to simulate the procedure, explore different scenarios, and make necessary adjustments before the actual surgery. This pre-surgical visualization could identify potential challenges and optimize the implant placement process.

For example, the computer-implemented method further comprises providing data for controlling a manufacturing of a physical drilling guide. The three-dimensional digital drilling guide model serves as a template for the physical drilling guide.

For example, the computer-implemented method further comprises controlling the manufacturing of the physical drilling guide using the data provided for controlling the manufacturing.

The physical drilling guide and/or the physical drilling guide model could be a tangible template used during dental implant surgery, created based on the three-dimensional digital drilling guide model. This physical guide may be manufactured using data generated from the digital model, ensuring that it accurately reflects the precise specifications required for the implant procedure. This physical guide might serve as a physical replica and/or copy of the three-dimensional digital drilling guide model, embodying the same through holes and drilling positions defined in the digital version. The physical drilling guide could be produced using advanced manufacturing techniques, such as 3D printing, milling or CNC milling, to ensure high precision and accuracy.

The physical drilling guide and/or the physical drilling guide model may have strategically placed through holes that align with the planned drilling positions on the patient's jaw. These holes might guide the surgeon’s drill, ensuring that the implants are placed at the correct angles, depths, sizes, and positions as specified by the digital model. For example, the physical drilling guide and/or the physical drilling guide model indicates the length of the drill. For example, the physical drilling guide and/or the physical drilling guide model may comprise means to indicate the length of the drill. The length may be within a range, e.g., in between 4 to 16 millimeters, or any other suitable range.

One advantage of the improved dental implant planning comprising the physical drilling guide and/or the physical drilling guide model could be that it may subsequently enhance the precision and accuracy of the implant placement. For example, by following the guide, the surgeon could reduce the risk of drilling errors, such as misalignment or incorrect depth or size, which could lead to complications or implant failure. This improved precision as a result of the improved dental implant planning might also improve the osseointegration process, where the implant may fuse effectively with the jawbone, e.g., resulting in a stable and durable foundation for the dental prosthetics.

Another advantage could be that the improved dental implant planning comprising the physical drilling guide and/or the physical drilling guide model could improve the efficiency of the surgical procedure as a result of the improved dental implant planning. For example, with a clear and accurate guide, the surgeon may perform the drilling process more quickly and with greater confidence, potentially reducing the overall time of the surgery as a result of the improved dental implant planning. This efficiency as a result of the improved dental implant planning might also decrease the time the patient spends under anesthesia, thereby lowering the associated risks.

Additionally, as a result of the improved dental implant planning using the physical drilling guide and/or the physical drilling guide model, patient satisfaction may be increased by ensuring a higher success rate of the implant procedure. Patients could experience fewer complications and better long-term outcomes as a result of the improved dental implant planning, leading to e.g., increased confidence in the treatment. The guide might also facilitate better communication between the dental team and the patient, as it provides a tangible reference that may be shown and explained.

For example, the physical drilling guide and/or the physical drilling guide model could be a critical tool in dental implant surgery, created as a physical copy of the three-dimensional digital drilling guide model. It may offer significant advantages, including enhanced precision, improved surgical efficiency, and increased patient satisfaction, by e.g., ensuring that implants are placed accurately and effectively.

For example, the computer-implemented method further comprises determining a patient specific panoramic curve descriptive of the curved form of a ridge of the patient’s jaw extending along a patient’s jaw bow using the three-dimensional digital jaw model. The area indicators of the jaw areas comprised by the implant library presets indicate jaw areas aligned on or alongside a generic panoramic curve, wherein the generic panoramic curve is descriptive of a generic curved form of a ridge of a generic patient’s jaw extending along a generic’s jaw bow. The determining of the one or more dental implant positions comprises a mapping of the generic panoramic curve to the patient specific panoramic curve. The mapping results in a modification of the indicated jaw areas. The determining of the set of dental implant parameters is based on the modified indicated jaw areas.

The patient specific panoramic curve may comprise a customized curve that accurately describes the unique shape and contour of an individual patient's jaw ridge. This curve may be derived from the three-dimensional digital jaw model of the patient, e.g., capturing the precise anatomical features of their jaw.

For example, the computer-implemented method may comprise determining a patient specific panoramic curve that describes the curved form of the ridge of the patient’s jaw. This ridge may extend along the patient's jaw bow, which may be the arc-shaped contour of the jawbone where teeth are positioned.

The generic panoramic curve may comprise a standardized curve that represents the typical shape and contour of a jaw ridge across a broader population. This generic curve could be achieved by measuring the panoramic curves of a representative set of patients or individuals. For example, a large sample of jaw scans from different individuals is analyzed to create an average or generic curve that may be used as a reference in the implant planning process.

The mapping process may involve aligning the generic panoramic curve to the patient specific panoramic curve. This mapping could help in adapting the generalized implant placement guidelines to the specific anatomical features of the patient’s jaw. For example, if the generic curve indicates typical areas for implant placement, mapping it to the patient's unique curve might reveal modified indicated jaw areas that are more suitable for the specific patient.

The modified indicated jaw areas may refer to the adjusted zones on the patient’s jaw where implants could be placed, based on the alignment of the generic curve with the patient specific curve. These modifications might ensure that the implant positions are tailored to fit the patient's unique jaw structure, leading to better alignment and stability of the implants.

By using a generic panoramic curve derived from a representative set of individuals, the method could provide a reliable starting point for implant placement. This generic curve may then be customized through mapping to match the patient’s specific jaw anatomy.

One advantage could be that by using a patient specific panoramic curve, this could enhance the accuracy of implant placement as a result of the improved dental implant planning, ensuring that the implants may be positioned in the most suitable locations for each individual. This accuracy may reduce the risk of complications such as misalignment or inadequate support for the implants. Furthermore, the customized approach could improve the aesthetic and functional outcomes, as the implants could be better integrated with the patient’s natural jaw structure.

Additionally, the process of mapping and modifying the indicated jaw areas could streamline the planning process. Dental professionals might use the generic curve as a baseline and then e.g., quickly adapt it to the patient’s specific needs, saving time and resources. This efficiency could lead to faster treatment planning and potentially shorter surgery times. By mapping a generic panoramic curve to this customized curve, the indicated jaw areas for implant placement could be modified to better fit the patient’s anatomy.

For example, the computer-implemented method further comprises using a first trained machine learning module to receive the preset indicator indicating the selected implant library preset. The first trained machine learning module is configured to provide the preset indicator as output in response to receiving the three-dimensional digital jaw model, the number of dental implants, and the plurality of implant library presets as input.

A machine learning module may refer to a software component that uses algorithms and statistical models to analyze and interpret data, making predictions or decisions based on this analysis. In the context of dental implant planning, a machine learning module could be designed to assist in selecting the appropriate implant library presets based on various inputs.

A trained machine learning module may be a machine learning module that has been trained on a large dataset to recognize patterns and make accurate predictions. For example, the computer-implemented method could comprise using a first trained machine learning module to receive the preset indicator, which indicates the selected implant library preset. This module might be trained by feeding it a large number of examples of three-dimensional digital jaw models, along with information about the number of dental implants and various implant library presets.

Once trained, the machine learning module could be configured to provide the preset indicator as output in response to receiving the three-dimensional digital jaw model, the number of dental implants, and the plurality of implant library presets as input. For example, when receiving a new patient's jaw model and the required number of implants, the module analyzes the data and suggest the most suitable implant library preset, streamlining the decision-making process for dental professionals.

As a possible advantage, it could enhance the accuracy and consistency of selecting implant library presets, potentially reducing the likelihood of human error. This accuracy might lead to better patient outcomes, as the implants could be placed more precisely according to the most suitable guidelines.

Furthermore, the use of a trained machine learning module could significantly increase efficiency. Dental professionals might save time by e.g., relying on the module's quick and accurate analysis, allowing them to e.g., focus on other aspects of patient care. Additionally, the module may continuously improve as it processes more data, becoming even more accurate and reliable over time.

The computer-implemented method may further comprise providing the first machine learning module to be trained. First training datasets for training the first machine learning module to be trained are provided, each first training dataset comprising a three-dimensional digital training jaw model, a training number of dental implants, the plurality of implant library presets, and a training preset indicator. The first machine learning module to be trained is trained using the training datasets.

Training datasets may refer to collections of data used to train a machine learning module, potentially enabling it to learn patterns and make accurate predictions. In the context of dental implant planning, training datasets could comprise comprehensive sets of information that include various examples of jaw models, implant numbers, library presets, and preset indicators. Each first training dataset might comprise a three-dimensional digital training jaw model, a training number of dental implants, the plurality of implant library presets, and a training preset indicator. These datasets may serve as the foundation for the machine learning module’s training process, allowing it to learn how to analyze and interpret the data effectively.

An advantage of using training datasets for the machine learning model could be that they may enable the machine learning module to learn from a wide variety of examples, e.g., enhancing its ability to generalize and make accurate predictions for new, unseen data. By training on these datasets, the module might become adept at identifying the most suitable implant library preset for any dataset it is given to when trained.

Additionally, using comprehensive training datasets could improve the module’s robustness and reliability. The more diverse and extensive the datasets, the better the module might perform in real-world applications, as it could have been exposed to a broad spectrum of scenarios during training. This exposure may lead to more accurate and consistent preset selection, reducing the likelihood of errors and improving patient outcomes.

For example, the computer-implemented method further comprises receiving planned positions of one or more artificial teeth relative to the three-dimensional digital jaw model.

Planned positions of one or more artificial teeth may refer to the specific locations within the jaw where artificial teeth may be intended to be placed. These positions may be determined based on careful analysis and planning to e.g., ensure optimal functionality and aesthetics. For example, the positions are determined by a computer-implemented method, a computer system, a computer program and/or a computer program product. For example, the positions are determined by a person skilled in the art. For example, the planned positions are determined by the dentist and/or the dental team. For example, in a computer-implemented dental implant procedure, the planned positions could be the exact coordinates and angles where the implants will be inserted into the jawbone and where the artificial teeth will be constructed on the implants, as indicated on the three-dimensional digital jaw model.

Artificial teeth could refer to prosthetic teeth designed to replace missing or damaged natural teeth. These may comprise various dental restorations such as crowns, bridges, or dentures, which are supported by the dental implants. For example, in the context of a dental implant procedure, artificial teeth might be the crowns that are attached to the implants to restore the patient's ability to chew and speak properly, as well as to enhance the appearance of their teeth.

For example, the computer-implemented method may further comprise receiving planned positions of one or more artificial teeth relative to the three-dimensional digital jaw model. This step could involve mapping out where each artificial tooth may be placed in relation to the existing teeth and jaw structure.

One advantage could be that the improved and precise planning could enhance the functionality and aesthetics of the dental restorations. By accurately mapping out the positions, the artificial teeth may align perfectly with the natural teeth as a result of the improved dental implant planning, e.g., ensuring a seamless appearance and proper bite alignment. This precision as a result of the improved dental implant planning might also reduce the risk of complications such as misalignment or discomfort, e.g., leading to improved patient satisfaction.

Moreover, receiving the planned positions of artificial teeth in advance could streamline the surgical procedure as a result of the improved dental implant planning. Dental professionals may use this improved dental implant planning comprising the planned positions of one or more artificial teeth to prepare and execute the implant placement more efficiently, potentially reducing the time the patient spends under anesthesia and the overall duration of the surgery. This efficiency might also minimize the risk of surgical errors and e.g., enhance the success rate of the implant procedure.

Additionally, having well-planned positions for artificial teeth could facilitate better communication between the dental team and the patient. Patients may be shown the planned outcomes, potentially helping them understand the procedure and set realistic expectations. This transparency might increase patient confidence and cooperation, contributing to a smoother treatment process.

For example, the computer-implemented method further comprises using a second trained machine learning module to receive the planned positions of the one or more artificial teeth. The second trained machine learning module is configured to provide the planned positions of the one or more artificial teeth as output in response to receiving the three-dimensional digital jaw model and the number of dental implants.

For example, the computer-implemented method further comprises providing the second machine learning module to be trained. Second training datasets for training the second machine learning module to be trained are provided, each second training dataset comprising a three-dimensional digital training jaw model and a training number of dental implants. The second machine learning module to be trained is trained using the training datasets.

One advantage of the second trained machine learning module could be that it could enhance the precision of planning the positions of artificial teeth. By leveraging the computational power and pattern recognition capabilities of the machine learning module, dental professionals might achieve more accurate placement of the implants, ensuring that the artificial teeth align correctly with the natural teeth and jaw structure.

Furthermore, the use of a trained machine learning module could streamline the planning process. This module may quickly analyze the inputs and generate optimal positions for the artificial teeth, reducing the time required for manual planning and allowing dental professionals to focus on other aspects of patient care. This efficiency as a result of the improved dental implant planning could also lead to shorter preparation times and more timely treatments for patients.

Additionally, the second trained machine learning module might contribute to better patient outcomes as a result of the improved dental implant planning. With more precise implant and artificial tooth positioning as a result of the improved dental implant planning, patients could e.g., experience fewer complications, such as misalignment or discomfort, potentially resulting in higher satisfaction with the procedure. Moreover, the improved accuracy might enhance the functionality and aesthetics of the dental restorations as a result of the improved dental implant planning, e.g., providing patients with better chewing function and a more natural-looking smile.

For example, the computer-implemented method further comprises using a third trained machine learning module to receive the number of dental implants. The third trained machine learning module is configured to provide the number of dental implants as output in response to receiving the three-dimensional digital jaw model as input.

For example, the computer-implemented method further comprises providing the third machine learning module to be trained. Third training datasets for training the third machine learning module to be trained are provided, each third training dataset comprising a three-dimensional digital training jaw model and a training number of dental implants. The third machine learning module to be trained is trained using the training datasets.

One advantage of the third trained machine learning module could be that it could enhance the accuracy of determining the number of dental implants needed for a particular patient. For example, by leveraging the module's advanced analytical capabilities, dental professionals might ensure that the appropriate number of implants is used, neither too few to compromise stability nor too many to cause unnecessary complexity or expense.

Furthermore, the use of this trained machine learning module could streamline the treatment planning process. The module may quickly analyze the three-dimensional digital jaw model and generate a precise recommendation for the number of dental implants, reducing the time required for manual assessment and planning. This efficiency might allow dental professionals to focus more on patient care and less on time-consuming calculations and estimations.

Additionally, the third trained machine learning module could contribute to better patient outcomes as a result of the improved dental implant planning. For example, with an accurately determined number of implants, patients might experience improved stability and functionality of their dental restorations. Properly planned implant placement could also enhance the aesthetics of the final outcome as a result of the improved dental implant planning, e.g., providing a more natural look and feel to the prosthetics.

For example, the set of dental implant parameters further define one or more dental implant sizes.

Dental implant sizes may refer to the dimensions of the implants themselves. These sizes might comprise the diameter and length of the implants. The sizes could range from diameters of 3.0 to 5.5 millimeters and lengths of 4 to 16 millimeters, providing flexibility to match the implant to the specific needs of the patient’s jawbone. Other ranges for size and length may also be possible.

An advantage could be that by e.g., selecting the correct implant sizes, this could be critical for achieving optimal osseointegration as a result of the improved dental implant planning, where the implant may fuse with the bone. Properly sized implants as a result of the improved dental implant planning may provide a stable foundation for the artificial teeth, potentially improving both the function and aesthetics of the dental restorations. This precision could reduce the likelihood of implant failure and the need for corrective surgeries, e.g., leading to better overall patient outcomes.

For example, the one or more relative angulations between at least two implants are determined such that at least two adjacent dental implants are arranged relative to each other with parallel orientations or mirrored orientations. Arranging the at least two adjacent dental implants relative to each other with mirrored orientations comprises a mirroring of a dental implant angles of one of the at least two adjacent dental implants using a mirror plane or mirror point arranged between the at least two adjacent dental implants.

Adjacent may refer to two or more implants that may be positioned next to each other in the jaw. These implants could be close in proximity, often to e.g., support a dental prosthesis such as a bridge or a set of crowns.

Parallel orientations may refer to the alignment of these adjacent dental implants so that they may be positioned in the same direction, with their axes e.g., being parallel or approximately parallel to each other. For example, if two or more adjacent dental implants are placed in parallel orientations, the angles at which they are inserted into the jaw could be the same, ensuring that the implants are aligned uniformly. For example, in this context, approximately parallel means that the angles at which the implants are inserted into the jaw are within a range of ±5 degrees relative to each other. This ensures that if one implant is placed at a specific angle, any adjacent implant should have an insertion angle that does not differ by more than 5 degrees from that of the first implant. This parallelism might be necessary for ensuring that the prosthetic device fits correctly and functions properly.

Mirrored orientations may refer to a specific alignment where the angles of adjacent dental implants are mirrored or approximately mirrored across a defined plane or point. For example, if two adjacent implants are placed with mirrored orientations, the angle of one implant might be the mirror image of the angle of the other implant across a "mirror plane" or "mirror point" located between them. This may be an exact mirror angle, or e.g., an approximate mirror angle, where the mirrored angle could deviate by ±5 degrees from the exact mirrored position. This approximation may help to accommodate minor variations while e.g., still achieving a symmetrical placement. This mirroring could be useful in achieving a symmetrical placement, potentially in aesthetic zones where the visual appearance of the implants may be crucial.

For example, a mirror plane is an imaginary flat surface that bisects the space between two adjacent implants, and across which the angles of the implants are mirrored. A mirror point may be a specific point between the implants from which the angles may be reflected.

The advantages of arranging dental implants in (approximate) parallel or mirrored orientations may be significant. Parallelism of dental implants which may be achieved as a result of the improved dental implant planning could ensure that the forces applied during chewing may be evenly distributed across the implants, potentially reducing the risk of implant failure and e.g., enhancing the stability of the dental prosthesis. This alignment which may be achieved as a result of the improved dental implant planning might also facilitate easier fitting and alignment of the prosthetic components, e.g., improving both the function and aesthetics of the final restoration.

Parallelism of implants or mirrored implants which may be achieved as a result of the improved dental implant planning may further facilitate the fabrication and fitting of dental prosthetics. For example, when implants are parallel or approximately parallel, the prosthetic components, such as bridges or dentures, might fit more accurately and securely onto the implants. This accurate fit could result in better functional outcomes and patient comfort, as the prosthesis could be less likely to move or cause irritation in the mouth.

Additionally, mirrored orientations or approximately mirrored orientations could enhance the symmetry and appearance of the dental implants, e.g., especially in visible areas such as the front of the mouth. This symmetry might provide a more natural and pleasing look, which could be important for patient satisfaction. Parallelism, in particular, may ensure even force distribution and facilitate better fitting of prosthetic components, e.g., leading to enhanced overall outcomes.

For example, the set of dental implant parameters are determined such that the one or more dental implants arranged within the patient’s jawbone according to the one or more dental implant parameters satisfy one or more of the following criteria: a vestibular minimum thickness of the patients’ jawbone in vestibular direction and an oral minimum thickness of the patients’ jawbone in oral direction being at least 1.5 mm, in particular at least 2 mm; a minimum implant distance between adjacent dental implants being at least 3 mm, in particular at least 4 mm; and a minimum implant-to-tooth distance between dental implants and roots of adjacent natural teeth being at least 1.5 mm, in particular at least 2 mm.

Vestibular thickness may refer to the thickness of the jawbone on the side facing the lips and cheeks, while vestibular minimum thickness could denote the smallest acceptable thickness in this area. Vestibular direction might describe the orientation towards the lips and cheeks as seen from the jawbone, the teeth and/or from the implants.

For example, the dental implant parameters could require that the vestibular minimum thickness of the patient's jawbone in the vestibular direction should be at least 1.5 mm, preferably at least 2 mm. This means that the bone should have a minimum thickness of 1.5 mm on the side facing the lips to provide adequate support for the implant.

Oral thickness may refer to the thickness of the jawbone on the side facing the tongue, while oral minimum thickness could denote the smallest acceptable thickness in this area. Oral direction might describe the orientation towards the tongue as seen from the jawbone, the teeth and/or from the implants.

For example, the implant parameters could require an oral minimum thickness of the jawbone in the oral direction to be at least 1.5 mm, preferably at least 2 mm. This may ensure that the bone has sufficient thickness on the side facing the tongue to support the implant.

Implant distance may refer to the distance between adjacent dental implants, while minimum implant distance could specify the smallest acceptable distance between these implants. For example, the parameters might require that the minimum implant distance between adjacent dental implants should be at least 3 mm, preferably at least 4 mm.

Implant-to-tooth distance may refer to the distance between a dental implant and the roots of adjacent natural teeth, while minimum implant-to-tooth distance could specify the smallest acceptable distance between an implant and a natural tooth.

For example, the implant parameters might require a minimum implant-to-tooth distance between dental implants and the roots of adjacent natural teeth to be at least 1.5 mm, preferably at least 2 mm. Roots and/or teeth roots may refer to the parts of natural teeth that are embedded in the jawbone and provide stability, while natural teeth could refer to the patient's existing, non-artificial teeth.

One advantage could be that by ensuring a vestibular and oral minimum thickness of at least 1.5 mm, preferably 2 mm, which may be achieved as a result of the improved dental implant planning, the stability and integration of the implants could be enhanced, e.g., reducing the risk of implant failure. Sufficient bone thickness in both the vestibular and oral directions may provide better support for the implants, potentially leading to improved long-term outcomes.

Maintaining a minimum implant distance of at least 3 mm, preferably 4 mm, which may be achieved as a result of the improved dental implant planning, could prevent the implants from interfering with each other, potentially ensuring that each implant has enough surrounding bone to support osseointegration. This spacing, which may be achieved as a result of the improved dental implant planning, might also facilitate better hygiene and reduce the risk of peri-implantitis, a condition that can cause implant failure.

Ensuring a minimum implant-to-tooth distance of at least 1.5 mm, preferably 2 mm, which may be achieved as a result of the improved dental implant planning, may protect the roots of adjacent natural teeth from damage during the implant procedure. This spacing, which may be achieved as a result of the improved dental implant planning, could also help maintain the health of the natural teeth and surrounding bone, leading to better overall oral health.

For example, the computer-implemented method further comprises generating the three-dimensional jaw model. The generating of the three-dimensional digital jaw model of the patient’s jaw comprises receiving a computed tomography scan of the patient’s jaw; receiving an intraoral scan of the patient’s jaw; combining the computed tomography scan with the intraoral scan and using the combination for the generating of the three-dimensional jaw model.

For example, the computed tomography scan of the patient’s jaw is a cone beam computed tomography scan of the patient’s jaw.

A computed tomography scan (CT scan) may refer to an imaging method that uses X-rays to create detailed cross-sectional images of the body. For example, in the context of dental implants, a CT scan of the patient's jaw could provide comprehensive information about the bone structure, including its density and contours.

For example, a cone beam computed tomography scan (CBCT scan) is a specific type of CT scan that uses a cone-shaped X-ray beam to create a three-dimensional image of the dental structures, soft tissues, nerve paths, and bone in the craniofacial region. For example, the method might involve using a CBCT scan to obtain a detailed view of the patient's jawbone.

For example, an intraoral scan refers to a digital impression of the inside of the mouth, captured using a handheld scanner. This scan could provide detailed images of the teeth and gums, e.g., allowing for the creation of accurate digital models of the patient’s dental structures. For example, an intraoral scan might capture the precise contours and alignment of the patient’s teeth and soft tissues.

Combining the computed tomography scan with the intraoral scan may involve integrating the detailed bone information from the CT scan with the details from the intraoral scan. This combination could create a comprehensive three-dimensional digital jaw model that may include both the internal and external structures of the patient's jaw. For example, by merging the CBCT scan with the intraoral scan, dental professionals might generate a detailed and accurate representation of the patient's entire oral anatomy.

An advantage of this combined approach which may be achieved as a result of the improved dental implant planning could be that it may provide a more complete and accurate representation of the patient’s jaw, thus e.g., enhancing the precision of dental implant planning. The detailed bone information from the CT scan, combined with the surface detail from the intraoral scan, might allow for better visualization of critical anatomical features, such as nerves and sinuses.

Furthermore, the integration of these imaging techniques could improve the fit and alignment of dental prosthetics which may be achieved as a result of the improved dental implant planning. By using a comprehensive digital model, dental professionals may design implants and prosthetics that may match the patient’s unique anatomy more closely, thus e.g., resulting in better functional and aesthetic outcomes.

Additionally, this method could streamline the treatment planning process which may be achieved as a result of the improved dental implant planning. With a detailed and accurate digital model, dental professionals might plan the entire procedure more efficiently and potentially feed this model to the computer-implemented method, e.g., reducing the time required for manual adjustments and potentially lowering the overall treatment time. This efficiency could enhance patient satisfaction by minimizing the duration and invasiveness of the treatment as a result of the improved dental implant planning, and also improve e.g., the precision of the computer-implemented method by integrating a highly detailed three-dimensional digital jaw model.

In another aspect, a computer program for determining one or more dental implant positions for one or more dental implants to be inserted into a patient’s jaw is disclosed. The computer program comprises program instructions. The program instructions are executable by a processor of a computer device to cause the computer device to receive a three-dimensional digital jaw model of the patient’s jaw. A number of dental implants to be inserted into the patient’s jaw is received. A preset indicator indicating an implant library preset selected from a plurality of implant library presets depending on the number of implants is received. The implant library presets are assigned to respective numbers of implants. The plurality of implant library presets comprise respective area indicators of jaw areas for insertion of the respective numbers of dental implants. Using the selected implant library preset and the three-dimensional digital jaw model, a set of dental implant parameters defining one or more dental implant positions relative to the three-dimensional digital jaw model are determined.

In another aspect, a computer device for determining one or more dental implant positions for one or more dental implants to be inserted into a patient’s jaw is disclosed. The computer device comprises a processor and a memory storing program instructions executable by the processor. Execution of the program instructions by the processor cause the computer device to receive a three-dimensional digital jaw model of the patient’s jaw. A number of dental implants to be inserted into the patient’s jaw is received. A preset indicator indicating an implant library preset selected from a plurality of implant library presets depending on the number of implants is received. The implant library presets are assigned to respective numbers of implants. The plurality of implant library presets comprise respective area indicators of jaw areas for insertion of the respective numbers of dental implants. Using the selected implant library preset and the three-dimensional digital jaw model, a set of dental implant parameters defining one or more dental implant positions relative to the three-dimensional digital jaw model are determined.

For example, a manufacturing system comprises the computer device. The manufacturing system further comprises a manufacturing device configured to manufacture a physical drilling template. Execution of the program instructions by the processor further cause the computer device to generate, using the dental implant parameters and the three-dimensional digital jaw model, a three-dimension digital drilling guide model. The three-dimensional digital drilling guide model comprises means for achieving the dental implant parameters for the insertion of the one or more dental implants. The means comprise one or more through holes defining drilling positions of drilling holes to be drilled into the patient’s jaw at the one or more dental implant positions. The drilling positions are to be drilled into patient’s jaw for the insertion of the one or more dental implants. Data for controlling the manufacturing of the physical drilling guide is provided. The three-dimensional digital drilling guide model serves as a template for the physical drilling guide. The manufacturing device is controlled to manufacture the physical drilling model using the data provided for controlling the manufacturing.

It is understood that examples of the computer-implemented method for determining one or more dental implant positions for one or more dental implants to be inserted into a patient’s jaw may be combined with the aspect of the computer program for determining one or more dental implant positions for one or more dental implants to be inserted into a patient’s jaw and/or the aspect of the computer device for determining one or more dental implant positions for one or more dental implants to be inserted into a patient’s jaw and/or the example of the manufacturing system, as long as the combined aspects and/or examples are not mutually exclusive.

It is understood that one or more of the aforementioned examples may be combined as long as the combined examples are not mutually exclusive.

### BRIEF DESCRIPTION OF THE DRAWINGS

In the following, examples are described in greater detail making reference to the drawings in which:

FIG. 1 is a schematic of a computer-implemented method for determining one or more dental implant positions for one or more dental implants to be inserted into a patient’s jaw,

FIG. 2 shows an example three-dimensional digital jaw model of a patient’s jaw comprising a patient specific panoramic curve indicating jaw areas,

FIG. 3 is an exemplary computer device for determining one or more dental implant positions for one or more dental implants to be inserted into a patient’s jaw.

FIG. 4 shows a manufacturing system comprising a computer device and a manufacturing device.

### DETAILED DESCRIPTION

In the following, similar elements are denoted by the same reference numerals.

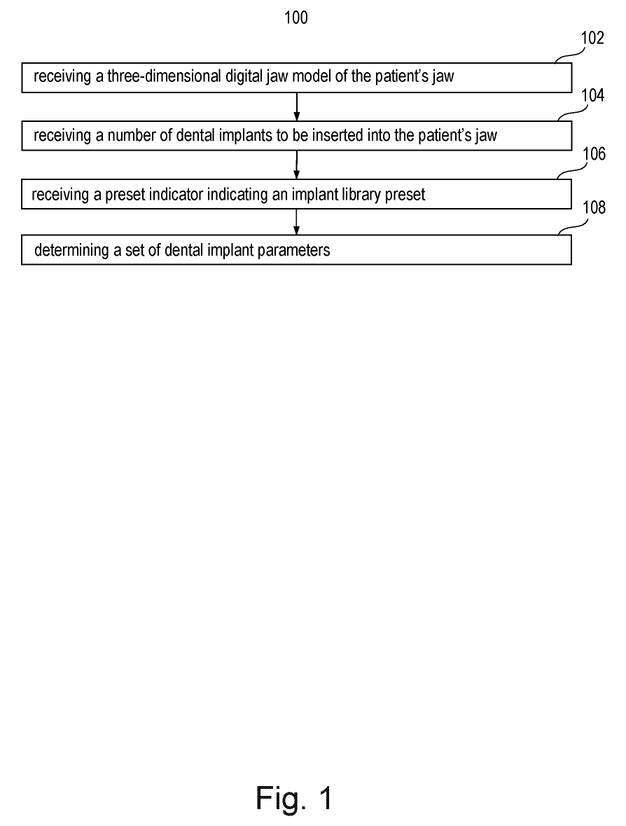

FIG. 1 shows a computer-implemented method 100 for determining one or more dental implant positions for one or more dental implants to be inserted into a patient’s jaw.

A three-dimensional digital jaw model of the patient’s jaw is received in block 102. For example, the digital three-dimensional jaw model is received from at least one medical scanner. For example, the at least one medical scanner is a cone-beam computed tomography, CBCT, scanner and an intraoral scanner. For example, the three-dimensional digital jaw model is received as raw data. For example, the three-dimensional digital jaw model of the patient’s jaw may be generated by the computer-implemented method. The generating may comprise receiving a cone-beam computed tomography scan of the patient’s jaw and an intraoral scan of the patient’s jaw from the respective medical scanner. Then, the computed tomography scan may be combined with the intraoral scan and the combination may be used for e.g., the generating of the three-dimensional jaw model.

A number of dental implants to be inserted into the patient’s jaw is received in block 104. For example, this may be a user input into the computer-implemented method or may be any other form of receiving the information by the computer-implemented method. In another example, the number is received from a third trained machine learning module. The third trained machine learning module is configured to provide the number of dental implants as output in response to receiving the three-dimensional digital jaw model as input. For example, the received number of dental implants is 4.

A preset indicator indicating an implant library preset selected from a plurality of implant library presets depending on the number of implants is received in block 106. For example, this may be a user input into the computer-implemented method or may be any other form of receiving the preset indicator by the computer-implemented method. The implant library presets are assigned to respective numbers of implants. In another example, the preset indicator is received from a first trained machine learning module. In another example, the preset indicator is automatically received from a computer-implemented method according to the number of dental implants. The first trained machine learning module may be configured to provide the preset indicator as output in response to receiving the three-dimensional digital jaw model, the number of dental implants, and the plurality of implant library presets as input. For example, a preset indicator indicating that an implant library preset is selected that is assigned to 4 dental implants is received.

For example, each implant library presets of the plurality of implant library presets corresponds to number of dental implants to be inserted into a patient’s jaw. There may be presets corresponding to 1 dental implants, 2 dental implants, 3 dental implants, 4 dental implants, 5 dental implants, 6 dental implants, 7 dental implants, 8 dental implants, 9 dental implants and/or 10 dental implants. In the example presented in the previous paragraph, the implant library preset corresponds to 4 dental implants.

The plurality of implant library presets comprise area indicators of jaw areas for insertion of the respective numbers of dental implants. For example, the area indicators specify optimal regions within the patient's jaw for the insertion of the dental implants based on various factors such as bone density, anatomical structures, and aesthetic considerations. For example, the area indicators for the preset corresponding to 4 dental implants might highlight the regions of the jawbone that may have sufficient bone volume and density to support the 4 implants, e.g., avoiding areas with critical anatomical structures such as nerves or sinus cavities.

Using the selected implant library preset and the three-dimensional digital jaw model, the computer-implemented method determines a set of dental implant parameters defining one or more dental implant positions relative to the three-dimensional digital jaw model in block 108. For example, these parameters may include the exact coordinates at which each of the four dental implants should be inserted into the jaw.

Several implanting techniques may be used in dental restoration, each e.g., tailored to the specific needs of the patient and the number of implants to be inserted into the jaw. For example, the set of dental implant parameters defining one or more dental implant positions relative to the three-dimensional digital jaw model may be determined according to an implanting technique. For example, the implant library presets are each assigned to a respective implanting technique, wherein each implanting technique is assigned to a respective number of dental implants. These techniques could comprise "All-on-4" for 4 dental implants, "All-on-6" for 6 dental implants, traditional dental bridges, and single-tooth implants for 1 dental implant.

In the example where the number of dental implant parameters is 4, the set of dental implant parameters may be determined according to the All-on-4 implanting technique. For example, the All-on-4 technique might determine that the implants should be placed at specific angles and positions to maximize support and functionality.

For example, the implant library presets further comprise one or more definitions of distances between at least two implants and/or one or more relative angulations between at least two implants, wherein the determined dental implant parameters further defining one or more dental implant angles for the one or more dental implants relative to the three-dimensional digital jaw model, and/or wherein the set of dental implant parameters further define one or more dental implant sizes.

For example, the All-on-4 technique may require two anterior implants to be placed at a depth of 12 mm with an angle of 30 degrees from the occlusal plane. These implants might be positioned in the jawbone to e.g., ensure maximum stability and support for the dental prosthesis. Furthermore, two posterior implants might be required to be placed at a depth of 10 mm with an angle of 45 degrees from the occlusal plane, e.g., ensuring they avoid critical anatomical structures like the sinus cavities while providing optimal support.

For example, the implant sizes might be determined based on the patient’s specific jaw anatomy and bone density. For example, the anterior implants might have a diameter of 4 mm and a length of 12 mm, while the posterior implants might have a diameter of 4.5 mm and a length of 10 mm.

For example, the method comprises generating, using the dental implant parameters, an output comprising a virtual implant positioning model. This virtual implant positioning model visually represents the one or more dental implant positions relative to the three-dimensional digital jaw model of the patient’s jaw. For example, the virtual model might display the precise locations and orientations of the four implants within the jaw, potentially allowing dental professionals to visualize and plan the implant placement accurately.

For example, the method may further comprise generating a three-dimensional digital drilling guide model. This model comprises means for achieving the dental implant parameters for the insertion of the one or more dental implants. The means may include one or more through holes defining drilling positions of drilling holes to be drilled into the patient’s jaw at the one or more dental implant positions. For example, the through holes in the digital drilling guide model may be precisely aligned with the determined implant positions to guide the surgical drill during the actual implant placement.

The three-dimensional digital drilling guide model may include through holes e.g., positioned at specific coordinates. For example, the first anterior implant hole might be positioned at coordinates (X1, Y1, Z1), the second anterior implant hole at (X2, Y2, Z2), the first posterior implant hole at (X3, Y3, Z3), and the second posterior implant hole at (X4, Y4, Z4), wherein the coordinates are in relation to the three-dimensional digital jaw model.

The computer-implemented method may further comprise providing data for controlling the manufacturing of a physical drilling guide. The three-dimensional digital drilling guide model serves as a template for the physical drilling guide. For example, the physical drilling guide may be produced using advanced manufacturing techniques such as 3D printing or CNC milling, ensuring high precision and accuracy.

For example, the physical drilling guide is utilized to precisely achieve the predetermined dental implant parameters within the patient’s jaw.

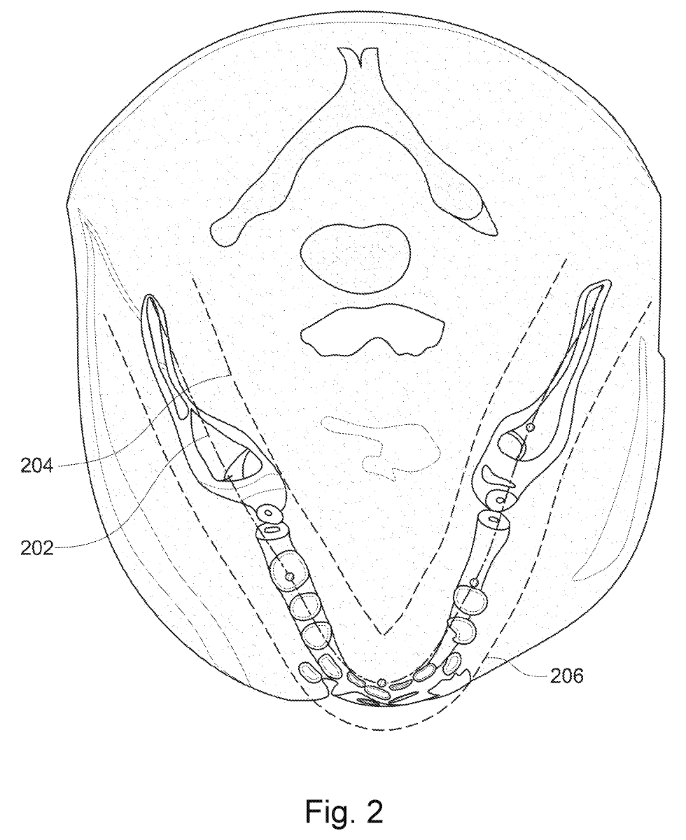

FIG. 2 shows an example three-dimensional digital jaw model of a patient’s jaw comprising a patient specific panoramic curve indicating jaw areas.

In FIG. 2, a patient specific panoramic curve 202 is depicted. The thickness of a jawbone in oral direction 204 may be 10mm, and the thickness of the jawbone in vestibular direction 206 may be 12mm. The patient-specific panoramic curve 202 might be formed by first acquiring the three-dimensional digital model of the patient's jaw.

For example, using this model, the computer-implemented method identifies the ridge of the patient’s jaw, which is the curved contour where the teeth are situated. The computer-implemented method might analyze the curvature and anatomical features of the jaw ridge to determine a customized panoramic curve that accurately represents the unique shape and alignment of the patient’s jawbone.

The process of determining the patient-specific panoramic curve 202 may involve e.g., comparing and mapping it to a generic panoramic curve. For example, the generic panoramic curve might be a standardized curve representing the typical shape and contour of a jaw ridge across a broader population. To e.g., create this generic curve, a large sample of jaw scans from different individuals could be analyzed. This analysis may result in an average or generic curve used e.g., as a reference in the implant planning process.

The mapping process might involve aligning the generic panoramic curve to the patient-specific panoramic curve. This alignment could help adapt the generalized implant placement guidelines to the specific anatomical features of the patient’s jaw. For example, if the generic curve indicates typical areas for implant placement, mapping it to the patient's unique curve might reveal modified indicated jaw areas that are more suitable for the specific patient.

For example, the modified indicated jaw areas may refer to the adjusted zones on the patient’s jaw where implants could be placed, based on the alignment of the generic curve with the patient-specific curve. These modifications might ensure that the implant positions are tailored to fit the patient's unique jaw structure.

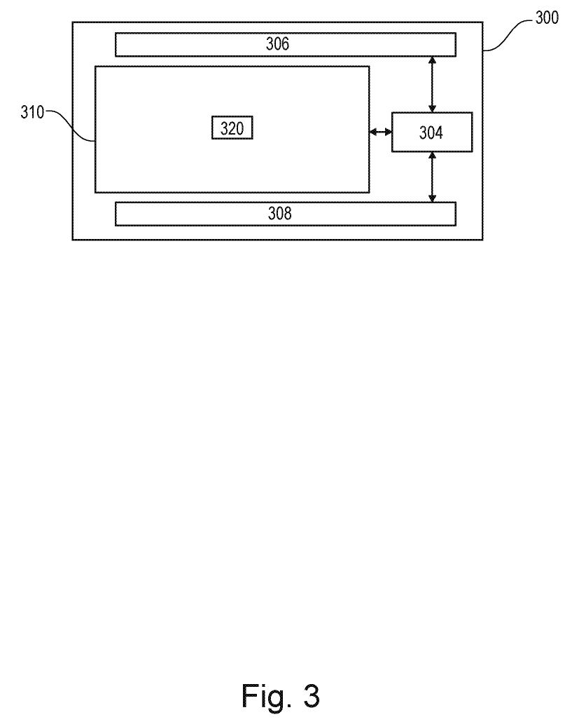

FIG. 3 displays an exemplary computer device 300 for determining one or more dental implant positions for one or more dental implants to be inserted into a patient’s jaw.

The computer device 300 is equipped with computing and hardware interfaces. The computer device 300 comprises a memory 310 comprising machine-executable instructions 320. The computer device 300 may further comprise hardware and/or software modules for receiving a three-dimensional digital jaw model of the patient’s jaw as in block 102. Software modules may be comprised by the machine-executable instructions 320. The hardware and/or software modules may further be configured to receive a number of dental implants to be inserted into the patient’s jaw as in block 104, to receive a preset indicator indicating an implant library preset as in block 106, and to determine a set of dental implant parameters as in block 108. The computer device 300 is intended to represent one or more computing units, which may be distributed. The computer device 300 is shown to comprise a computing system 304. The computing system 304 is intended to represent one or more computing systems. The computer device 300 is further shown to include an optional hardware interface 306. The hardware interface may enable the computer device 300 to send and receive data from external components. The computer device 300 is further shown to be in communication with an optional user interface 308. The computer device 300 may also comprise, for example, a display device. This could include, for example, a two-dimensional computer display, a touch screen, a virtual reality system, and an augmented reality system, or it may be in a form that it provides a virtual reality stream comprising a live camera view to a virtual reality headset. The hardware interface may also offer means to connect a headtracking device.

The computing system 304 may comprise a processor. The computing system 304 is further shown to be in communication with the memory 310. The memory 310 is intended to represent various types of memory that the computing system 304 may have access to. In one example, the memory 310 is a non-volatile storage medium.

The memory 310 is configured to comprise the machine-executable instructions 320. The machine-executable instructions 320 may enable the computing system 304 to perform various numerical and computational tasks. The machine-executable instructions 320 may also enable the computing system 304 to send and receive data from external components via the hardware interface 306. Execution of the machine-executable instructions 320 by the computing system 304 cause the computing system 304 to execute a method similar or equal to the computer-implemented method 100.

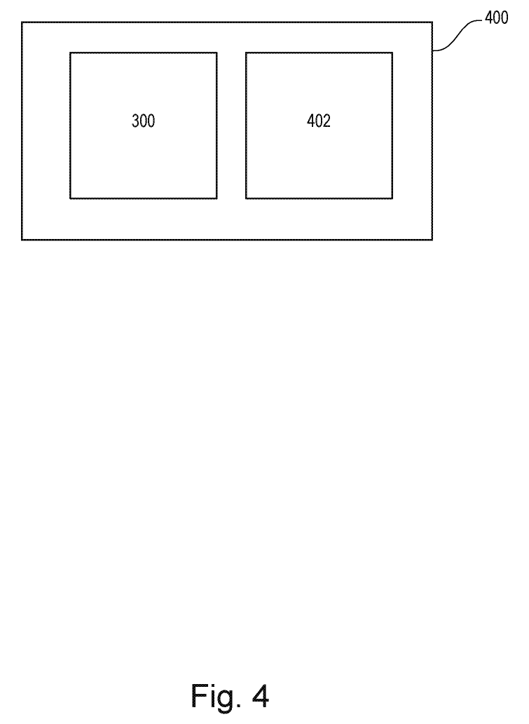

FIG. 4 shows a manufacturing system comprising a computer device 300 and a manufacturing device 402.

While the invention has been illustrated and described in detail in the drawings and foregoing description, such illustration and description are to be considered illustrative or exemplary and not restrictive; the invention is not limited to the disclosed examples.

As will be appreciated by one skilled in the art, aspects of the present invention may be embodied as an apparatus, method, computer program or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon. A computer program comprises the computer executable code or "program instructions".

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example, a data may be retrieved over a modem, over the internet, or over a local area network. Computer executable code embodied on a computer readable medium may be transmitted using any appropriate medium, including but not limited to wireless, wireline, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a processor. ‘Computer storage’ or ‘storage’ is a further example of a computer-readable storage medium. Computer storage is any non-volatile computer-readable storage medium. In some embodiments computer storage may also be computer memory or vice versa.

A ‘processor’ as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object oriented programming language such as Java, Smalltalk, C++ or the like and conventional procedural programming languages, such as the "C" programming language or similar programming languages and compiled into machine executable instructions. In some instances the computer executable code may be in the form of a high level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Generally, the program instructions can be executed on one processor or on several processors. In the case of multiple processors, they can be distributed over several different entities like clients, servers etc. Each processor could execute a portion of the instructions intended for that entity. Thus, when referring to a system or process involving multiple entities, the computer program or program instructions are understood to be adapted to be executed by a processor associated or related to the respective entity.

Aspects of the present invention are described with reference to flowchart illustrations and/or block diagrams of methods, apparatus (systems) and computer program products according to embodiments of the invention. It will be understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The invention may also be described using the following clauses:

Clause 1: A computer-implemented method for determining one or more dental implant positions for one or more dental implants to be inserted into a patient’s jaw, the method comprising: receiving a three-dimensional digital jaw model of the patient’s jaw; receiving a number of dental implants to be inserted into the patient’s jaw; receiving a preset indicator indicating an implant library preset selected from a plurality of implant library presets depending on the number of implants, wherein the implant library presets are assigned to respective numbers of implants, the plurality of implant library presets comprising area indicators of jaw areas for insertion of the respective numbers of dental implants; and determining, using the selected implant library preset and the three-dimensional digital jaw model, a set of dental implant parameters defining one or more dental implant positions relative to the three-dimensional digital jaw model.

Clause 2: The computer-implemented method of clause 1, wherein the implant library presets further comprise one or more definitions of distances between at least two implants.

Clause 3: The computer-implemented method of any of the previous clauses, wherein the implant library presets further comprise one or more relative angulations between at least two implants, the determined dental implant parameters further defining one or more dental implant angles for the one or more dental implants relative to the three-dimensional digital jaw model.

Clause 4: The computer-implemented method of any of the previous clauses, further comprising: generating, using the dental implant parameters, an output comprising a virtual implant positioning model, wherein the virtual implant positioning model visually represents the one or more dental implant positions relative to the three-dimensional digital jaw model of the patient’s jaw.

Clause 5: The computer-implemented method of any of the previous clauses, further comprising: generating, using the dental implant parameters and the three-dimensional digital jaw model, a three-dimensional digital drilling guide model, wherein the three-dimensional digital drilling guide model comprises means for achieving the dental implant parameters for the insertion of the one or more dental implants, wherein the means comprise one or more through holes defining drilling positions of drilling holes to be drilled into the patient’s jaw at the one or more dental implant positions, wherein the drilling positions are to be drilled into patient’s jaw for the insertion of the one or more dental implants.

Clause 6: The computer-implemented method of clause 5, further comprising: providing data for controlling a manufacturing of a physical drilling guide, the three-dimensional digital drilling guide model serving as a template for the physical drilling guide.

Clause 7: The computer-implemented method of clause 6, further comprising: controlling the manufacturing of the physical drilling guide using the data provided for controlling the manufacturing.

Clause 8: The computer-implemented method of any of the previous clauses, further comprising: determining a patient specific panoramic curve descriptive of the curved form of a ridge of the patient’s jaw extending along a patient’s jaw bow using the three-dimensional digital jaw model, wherein the area indicators of the jaw areas comprised by the implant library presets indicate jaw areas aligned on or alongside a generic panoramic curve, wherein the generic panoramic curve is descriptive of a generic curved form of a ridge of a generic patient’s jaw extending along a generic’s jaw bow, wherein the determining of the one or more dental implant positions comprises a mapping of the generic panoramic curve to the patient specific panoramic curve, the mapping resulting in a modification of the indicated jaw areas, the determining of the set of dental implant parameters being based on the modified indicated jaw areas.

Clause 9: The computer-implemented method of any of the previous clauses, using a first trained machine learning module to receive the preset indicator indicating the selected implant library preset, the first trained machine learning module being configured to provide the preset indicator as output in response to receiving the three-dimensional digital jaw model, the number of dental implants, and the plurality of implant library presets as input.

Clause 10: The computer-implemented method of clause 9, further comprising: providing the first machine learning module to be trained; providing first training datasets for training the first machine learning module to be trained, each first training dataset comprising a three-dimensional digital training jaw model, a training number of dental implants, the plurality of implant library presets, and a training preset indicator; training the first machine learning module to be trained using the training datasets.

Clause 11: The computer-implemented method of any of the previous clauses, further comprising receiving planned positions of one or more artificial teeth relative to the three-dimensional digital jaw model.

Clause 12: The computer-implemented method of clause 11, using a second trained machine learning module to receive the planned positions of the one or more artificial teeth, the second trained machine learning module being configured to provide the planned positions of the one or more artificial teeth as output in response to receiving the three-dimensional digital jaw model and the number of dental implants.

Clause 13: The computer-implemented method of any of the previous clauses, using a third trained machine learning module to receive the number of dental implants, the third trained machine learning module being configured to provide the number of dental implants as output in response to receiving the three-dimensional digital jaw model as input.

Clause 14: The computer-implemented method of any of the previous clauses, wherein the set of dental implant parameters further define one or more dental implant sizes.

Clause 15: The computer-implemented method of any of clause 3 to 14, wherein the one or more relative angulations between at least two implants are determined such that at least two adjacent dental implants are arranged relative to each other with parallel orientations or mirrored orientations, wherein arranging the at least two adjacent dental implants relative to each other with mirrored orientations comprises a mirroring of a dental implant angles of one of the at least two adjacent dental implants using a mirror plane or mirror point arranged between the at least two adjacent dental implants.

Clause 16: The computer-implemented method of clause 14, wherein the set of dental implant parameters are determined such that the one or more dental implants arranged within the patient’s jawbone according to the one or more dental implant parameters satisfy one or more of the following criteria: a vestibular minimum thickness of the patients’ jawbone in vestibular direction and an oral minimum thickness of the patients’ jawbone in oral direction being at least 1.5 mm, in particular at least 2 mm; a minimum implant distance between adjacent dental implants being at least 3 mm, in particular at least 4 mm; and a minimum implant-to-tooth distance between dental implants and roots of adjacent natural teeth being at least 1.5 mm, in particular at least 2 mm.

Clause 17: The computer-implemented method of any of the previous clauses, further comprising: generating the three-dimensional jaw model, wherein generating the three-dimensional digital jaw model of the patient’s jaw comprises: receiving a computed tomography scan of the patient’s jaw; receiving an intraoral scan of the patient’s jaw; combining the computed tomography scan with the intraoral scan and using the combination for the generating of the three-dimensional jaw model.

Clause 18: A computer program for determining one or more dental implant positions for one or more dental implants to be inserted into a patient’s jaw, the computer program comprising program instructions, the program instructions being executable by a processor of a computer device to cause the computer device to: receive a three-dimensional digital jaw model of the patient’s jaw; receive a number of dental implants to be inserted into the patient’s jaw; receive a preset indicator indicating an implant library preset selected from a plurality of implant library presets depending on the number of implants, wherein the implant library presets are assigned to respective numbers of implants, the plurality of implant library presets comprising respective area indicators of jaw areas for insertion of the respective numbers of dental implants; and determine, using the selected implant library preset and the three-dimensional digital jaw model, a set of dental implant parameters defining one or more dental implant positions relative to the three-dimensional digital jaw model.

Clause 19: A computer device for determining one or more dental implant positions for one or more dental implants to be inserted into a patient’s jaw, the computer device comprising a processor and a memory storing program instructions executable by the processor, execution of the program instructions by the processor causing the computer device to: receive a three-dimensional digital jaw model of the patient’s jaw; receive a number of dental implants to be inserted into the patient’s jaw; receive a preset indicator indicating an implant library preset selected from a plurality of implant library presets depending on the number of implants, wherein the implant library presets are assigned to respective numbers of implants, the plurality of implant library presets comprising respective area indicators of jaw areas for insertion of the respective numbers of dental implants; and determine, using the selected implant library preset and the three-dimensional digital jaw model, a set of dental implant parameters defining one or more dental implant positions relative to the three-dimensional digital jaw model.

Clause 20: A manufacturing system comprising the computer device of clause 19, the manufacturing system further comprising a manufacturing device configured to manufacture a physical drilling template, execution of the program instructions by the processor further causing the computer device to: generate, using the dental implant parameters and the three-dimensional digital jaw model, a three-dimension digital drilling guide model, wherein the three-dimensional digital drilling guide model comprises means for achieving the dental implant parameters for the insertion of the one or more dental implants, wherein the means comprise one or more through holes defining drilling positions of drilling holes to be drilled into the patient’s jaw at the one or more dental implant positions, wherein the drilling positions are to be drilled into patient’s jaw for the insertion of the one or more dental implants; provide data for controlling the manufacturing of the physical drilling guide, the three-dimensional digital drilling guide model serving as a template for the physical drilling guide; control the manufacturing device to manufacture the physical drilling model using the data provided for controlling the manufacturing.

### REFERENCE SIGNS LIST

100 Computer-implemented method for determining one or more dental implant positions

102 Block for receiving a three-dimensional digital jaw model of the patient’s jaw

104 Block for receiving a number of dental implants to be inserted into the patient’s jaw

106 Block for receiving a preset indicator indicating an implant library preset

108 Block for determining a set of dental implant parameters

202 Patient-specific panoramic curve

204 Thickness of the jawbone in oral direction

206 Thickness of the jawbone in vestibular direction

300 Computer device

304 Computing system

306 Hardware interface

308 User interface

310 Memory

320 Machine-executable instructions

402 Manufacturing device

1. A computer-implemented method for determining one or more dental implant positions for one or more dental implants to be inserted into a patient’s jaw, the method comprising:
receiving a three-dimensional digital jaw model of the patient’s jaw;
receiving a number of dental implants to be inserted into the patient’s jaw;
receiving a preset indicator indicating an implant library preset selected from a plurality of implant library presets depending on the number of implants, wherein the implant library presets are assigned to respective numbers of implants, the plurality of implant library presets comprising area indicators of jaw areas for insertion of the respective numbers of dental implants; and
determining, using the selected implant library preset and the three-dimensional digital jaw model, a set of dental implant parameters defining one or more dental implant positions relative to the three-dimensional digital jaw model.

2. The computer-implemented method of claim 1, wherein the implant library presets further comprise one or more definitions of distances between at least two implants.

3. The computer-implemented method of claim 1, wherein the implant library presets further comprise one or more relative angulations between at least two implants, the determined dental implant parameters further defining one or more dental implant angles for the one or more dental implants relative to the three-dimensional digital jaw model.

4. The computer-implemented method of claim 1, further comprising:
generating, using the dental implant parameters, an output comprising a virtual implant positioning model, wherein the virtual implant positioning model visually represents the one or more dental implant positions relative to the three-dimensional digital jaw model of the patient’s jaw.

5. The computer-implemented method of claim 1, further comprising:
generating, using the dental implant parameters and the three-dimensional digital jaw model, a three-dimensional digital drilling guide model, wherein the three-dimensional digital drilling guide model comprises means for achieving the dental implant parameters for the insertion of the one or more dental implants, wherein the means comprise one or more through holes defining drilling positions of drilling holes to be drilled into the patient’s jaw at the one or more dental implant positions, wherein the drilling positions are to be drilled into patient’s jaw for the insertion of the one or more dental implants.

6. The computer-implemented method of claim 5, further comprising:
providing data for controlling a manufacturing of a physical drilling guide, the three-dimensional digital drilling guide model serving as a template for the physical drilling guide.

7. The computer-implemented method of claim 6, further comprising:
controlling the manufacturing of the physical drilling guide using the data provided for controlling the manufacturing.

8. The computer-implemented method of claim 1, further comprising:
determining a patient specific panoramic curve descriptive of the curved form of a ridge of the patient’s jaw extending along a patient’s jaw bow using the three-dimensional digital jaw model, wherein the area indicators of the jaw areas comprised by the implant library presets indicate jaw areas aligned on or alongside a generic panoramic curve, wherein the generic panoramic curve is descriptive of a generic curved form of a ridge of a generic patient’s jaw extending along a generic’s jaw bow, wherein the determining of the one or more dental implant positions comprises a mapping of the generic panoramic curve to the patient specific panoramic curve, the mapping resulting in a modification of the indicated jaw areas, the determining of the set of dental implant parameters being based on the modified indicated jaw areas.

9. The computer-implemented method of claim 1, using a first trained machine learning module to receive the preset indicator indicating the selected implant library preset, the first trained machine learning module being configured to provide the preset indicator as output in response to receiving the three-dimensional digital jaw model, the number of dental implants, and the plurality of implant library presets as input.

10. The computer-implemented method of claim 9, further comprising:
providing the first machine learning module to be trained;
providing first training datasets for training the first machine learning module to be trained, each first training dataset comprising a three-dimensional digital training jaw model, a training number of dental implants, the plurality of implant library presets, and a training preset indicator;
training the first machine learning module to be trained using the training datasets.

11. The computer-implemented method of claim 1, further comprising receiving planned positions of one or more artificial teeth relative to the three-dimensional digital jaw model.

12. The computer-implemented method of claim 11, using a second trained machine learning module to receive the planned positions of the one or more artificial teeth, the second trained machine learning module being configured to provide the planned positions of the one or more artificial teeth as output in response to receiving the three-dimensional digital jaw model and the number of dental implants.

13. The computer-implemented method of claim 1, using a third trained machine learning module to receive the number of dental implants, the third trained machine learning module being configured to provide the number of dental implants as output in response to receiving the three-dimensional digital jaw model as input.

14. The computer-implemented method of claim 1, wherein the set of dental implant parameters further define one or more dental implant sizes.

15. The computer-implemented method of claim 3, wherein the one or more relative angulations between at least two implants are determined such that at least two adjacent dental implants are arranged relative to each other with parallel orientations or mirrored orientations, wherein arranging the at least two adjacent dental implants relative to each other with mirrored orientations comprises a mirroring of a dental implant angles of one of the at least two adjacent dental implants using a mirror plane or mirror point arranged between the at least two adjacent dental implants.

16. The computer-implemented method of claim 14, wherein the set of dental implant parameters are determined such that the one or more dental implants arranged within the patient’s jawbone according to the one or more dental implant parameters satisfy one or more of the following criteria:
a vestibular minimum thickness of the patients’ jawbone in vestibular direction and an oral minimum thickness of the patients’ jawbone in oral direction being at least 1.5 mm, in particular at least 2 mm;
a minimum implant distance between adjacent dental implants being at least 3 mm, in particular at least 4 mm; and
a minimum implant-to-tooth distance between dental implants and roots of adjacent natural teeth being at least 1.5 mm, in particular at least 2 mm.

17. The computer-implemented method of claim 1, further comprising:
generating the three-dimensional jaw model, wherein generating the three-dimensional digital jaw model of the patient’s jaw comprises:
receiving a computed tomography scan of the patient’s jaw;
receiving an intraoral scan of the patient’s jaw;
combining the computed tomography scan with the intraoral scan and using the combination for the generating of the three-dimensional jaw model.

18. A computer program for determining one or more dental implant positions for one or more dental implants to be inserted into a patient’s jaw, the computer program comprising program instructions, the program instructions being executable by a processor of a computer device to cause the computer device to:
receive a three-dimensional digital jaw model of the patient’s jaw;
receive a number of dental implants to be inserted into the patient’s jaw;
receive a preset indicator indicating an implant library preset selected from a plurality of implant library presets depending on the number of implants, wherein the implant library presets are assigned to respective numbers of implants, the plurality of implant library presets comprising respective area indicators of jaw areas for insertion of the respective numbers of dental implants; and
determine, using the selected implant library preset and the three-dimensional digital jaw model, a set of dental implant parameters defining one or more dental implant positions relative to the three-dimensional digital jaw model.

19. A computer device for determining one or more dental implant positions for one or more dental implants to be inserted into a patient’s jaw, the computer device comprising a processor and a memory storing program instructions executable by the processor, execution of the program instructions by the processor causing the computer device to:
receive a three-dimensional digital jaw model of the patient’s jaw;
receive a number of dental implants to be inserted into the patient’s jaw;
receive a preset indicator indicating an implant library preset selected from a plurality of implant library presets depending on the number of implants, wherein the implant library presets are assigned to respective numbers of implants, the plurality of implant library presets comprising respective area indicators of jaw areas for insertion of the respective numbers of dental implants; and
determine, using the selected implant library preset and the three-dimensional digital jaw model, a set of dental implant parameters defining one or more dental implant positions relative to the three-dimensional digital jaw model.

20. A manufacturing system comprising the computer device of claim 19, the manufacturing system further comprising a manufacturing device configured to manufacture a physical drilling template, execution of the program instructions by the processor further causing the computer device to:
generate, using the dental implant parameters and the three-dimensional digital jaw model, a three-dimension digital drilling guide model, wherein the three-dimensional digital drilling guide model comprises means for achieving the dental implant parameters for the insertion of the one or more dental implants, wherein the means comprise one or more through holes defining drilling positions of drilling holes to be drilled into the patient’s jaw at the one or more dental implant positions, wherein the drilling positions are to be drilled into patient’s jaw for the insertion of the one or more dental implants;
provide data for controlling the manufacturing of the physical drilling guide, the three-dimensional digital drilling guide model serving as a template for the physical drilling guide;
control the manufacturing device to manufacture the physical drilling model using the data provided for controlling the manufacturing.
